(19) United States
(12) Patent Application Publication
(10) Pub. No.: US 2026/0060631 A1
(43) Pub. Date: Mar. 5, 2026

(54) CT APPARATUS, OPERATION METHOD OF CT APPARATUS, OPERATION PROGRAM OF CT APPARATUS, AND IMAGE DISPLAY APPARATUS

(71) Applicant: FUJIFILM Corporation, Tokyo (JP)

(72) Inventor: Jun MASUMOTO, Tokyo (JP)

(73) Assignee: FUJIFILM Corporation, Tokyo (JP)

(21) Appl. No.: 19/382,289

(22) Filed: Nov. 7, 2025

Related U.S. Application Data
(63) Continuation of application No. PCT/JP2024/016736, filed on Apr. 30, 2024.

(30) Foreign Application Priority Data
May 10, 2023 (JP) 2023-078080

(51) Int. Cl. A61B 6/00; A61B 6/03; A61B 6/42

(52) U.S. Cl. CPC A61B 6/5205; A61B 6/032; A61B 6/4241

(57) ABSTRACT

A CT apparatus includes a photon-counting radiation detector, and a processor, in which the CT apparatus performs imaging in which projection data of radiation transmitted through a subject at a plurality of circumferential positions around the subject is detected by the radiation detector, and generates a tomographic image of the subject by reconstructing the projection data, and the processor detects a specific region that meets a predetermined condition by performing image analysis on a first tomographic image obtained by reconstructing the projection data, and generates a second tomographic image of a second imaging range that is narrower than a first imaging range of the first tomographic image and that includes the specific region.

### CROSS-REFERENCE TO RELATED APPLICATIONS

This application is a continuation application of International Application No. PCT/JP2024/016736, filed Apr. 30, 2024, the disclosure of which is incorporated herein by reference in its entirety. Further, this application claims priority from Japanese Patent Application No. 2023-078080, filed on May 10, 2023, the disclosures of each are incorporated herein by reference in their entirety.

### BACKGROUND

### 1. Technical Field

The disclosed technology relates to a CT apparatus, an operation method of a CT apparatus, an operation program of a CT apparatus, and an image display apparatus.

### 2. Description of the Related Art

JP2005-198798A discloses an image processing apparatus including an analysis unit that analyzes information on a position and a range of a region of interest from a radiation image, and a condition determination unit that determines imaging conditions based on the information analyzed by the analysis unit.

### SUMMARY

A computed tomography (CT) apparatus (hereinafter, referred to as a photon counting computed tomography (PCCT) apparatus) comprising a photon-counting radiation detector has been developed. The PCCT apparatus can obtain a high-resolution image that is a high-resolution tomographic image, as compared with the CT apparatus in the related art and, moreover, a material discrimination image in which materials having different attenuation coefficients of radiation are discriminated and visualized. The high-resolution image or the material discrimination image has potential for enabling diagnostic support that has been impossible in the related art, and further utilization is expected in the future.

However, the high-resolution image and the material discrimination image have a very large amount of data as compared with the tomographic image of the CT apparatus in the related art, and there is a concern that the capacity of an image database (DB) such as a picture archiving and communication system (PACS) is strained.

The disclosed technology provides a CT apparatus, an operation method of a CT apparatus, an operation program of a CT apparatus, and an image display apparatus that enable utilization of a high-resolution image or a material discrimination image that can be acquired by a photon-counting radiation detector while suppressing an amount of data to be stored.

The disclosed technology provides a CT apparatus comprising: a photon-counting radiation detector; and a processor, in which the CT apparatus performs imaging in which projection data of radiation transmitted through a subject at a plurality of circumferential positions around the subject is detected by the radiation detector, and generates a tomographic image of the subject by reconstructing the projection data, and the processor detects a specific region that meets a predetermined condition by performing image analysis on a first tomographic image obtained by reconstructing the projection data, and generates a second tomographic image of a second imaging range that is narrower than a first imaging range of the first tomographic image and that includes the specific region.

It is preferable that the processor generate the first tomographic image by reconstructing first projection data obtained by performing the imaging in a first imaging mode as the projection data, detect the specific region by performing the image analysis on the first tomographic image, and generate the second tomographic image by reconstructing second projection data obtained by performing the imaging in a second imaging mode different from the first imaging mode.

It is preferable that in a case in which the second tomographic image is a high-resolution image having higher resolution than the first tomographic image, the second projection data be projection data for high resolution having higher resolution than the first projection data, and in a case in which the second tomographic image is a material discrimination image capable of discriminating and visualizing a plurality of materials having different attenuation coefficients of the radiation, the second projection data be projection data for material discrimination capable of being reconstructed into the material discrimination image.

It is preferable that the processor determine imaging conditions for the second imaging mode based on the specific region detected from the first tomographic image.

It is preferable that the processor reconstruct the second tomographic image using a part of the projection data used for reconstructing the first tomographic image.

It is preferable that in a case in which the second tomographic image is a high-resolution image having higher resolution than the first tomographic image, the projection data be projection data for high resolution capable of being reconstructed into the second tomographic image, and in a case in which the second tomographic image is a material discrimination image capable of discriminating and visualizing a plurality of materials having different attenuation coefficients of the radiation, the projection data be projection data for material discrimination capable of being reconstructed into the material discrimination image.

It is preferable that in a case in which the projection data is the projection data for high resolution or the projection data for material discrimination, the processor perform, in a case of generating the first tomographic image, data reduction processing on the projection data and then reconstruct the first tomographic image based on the projection data on which the data reduction processing has been performed.

It is preferable that the predetermined condition be that the specific region includes a specific organ or an abnormal part.

It is preferable that the processor perform the image analysis using a machine learning model.

It is preferable that the processor generate, in a case in which a plurality of the specific regions are detected, the second tomographic image for each specific region.

It is preferable that the processor generate a third tomographic image of a third imaging range that is narrower than the second imaging range of the second tomographic image based on a result of the image analysis of the second tomographic image.

Further, the disclosed technology provides an operation method of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data, the operation method comprising: via the processor, detecting a specific region that meets a predetermined condition by performing image analysis on a first tomographic image obtained by reconstructing the projection data; and generating a second tomographic image of a second imaging range that is narrower than a first imaging range of the first tomographic image and that includes the specific region.

Further, the disclosed technology provides an operation program of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data, the operation program causing the processor to execute: a step of detecting a specific region that meets a predetermined condition by performing image analysis on a first tomographic image obtained by reconstructing the projection data; and a step of generating a second tomographic image of a second imaging range that is narrower than a first imaging range of the first tomographic image and that includes the specific region.

Further, the disclosed technology provides an image display apparatus that displays the first tomographic image and the second tomographic image obtained by the CT apparatus, the image display apparatus comprising: a display control processor that controls display of the image display apparatus, in which the display control processor displays, in a case in which the first tomographic image is displayed on a display, an indicator indicating the second imaging range in the first tomographic image, and displays, in a case in which a designation operation is performed on the indicator, the second tomographic image.

According to the disclosed technology, it is possible to utilize the high-resolution image or the material discrimination image that can be acquired by the photon-counting radiation detector while suppressing the amount of data to be stored.

### BRIEF DESCRIPTION OF THE DRAWINGS

FIG. 1 is a schematic diagram of a tomographic imaging system.

FIG. 2 is a schematic diagram of a CT apparatus.

FIG. 3 is a diagram showing a photon-counting detector.

FIG. 4 is a diagram showing an integrating-type detector in the related art.

FIG. 5 is a schematic diagram of a discriminator.

FIG. 6 is a diagram schematically showing a function of the discriminator.

FIG. 7 is a diagram showing an example of an energy distribution of X-rays.

FIG. 8 is a diagram showing an example of an attenuation coefficient.

FIG. 9 is a diagram showing processing of a processor according to a first embodiment.

FIG. 10 is a flowchart showing a processing procedure of the first embodiment.

FIG. 11 is a table showing an example of specifications of a first tomographic image and a second tomographic image.

FIG. 12 is a table showing an example of imaging conditions of each imaging mode.

FIG. 13 is a diagram showing a specific example of the first tomographic image and the second tomographic image.

FIG. 14 is a diagram showing another specific example of the first tomographic image and the second tomographic image.

FIG. 15 is a diagram showing image analysis by a machine learning model.

FIG. 16 is a diagram showing an example in a case in which there are a plurality of specific regions.

FIG. 17 is a diagram showing an example in which a third tomographic image is acquired by performing image analysis on the second tomographic image.

FIG. 18 is a diagram showing processing of a processor according to a second embodiment.

FIG. 19 is a flowchart showing a processing procedure of the second embodiment.

FIG. 20 is a diagram showing imaging conditions in the second embodiment.

FIG. 21 is a diagram showing a hardware configuration of an image display apparatus.

FIG. 22 is a diagram showing an example of an image display screen on which the first tomographic image is displayed.

FIG. 23 is a diagram showing an example of a state in which the second tomographic image is displayed on the image display screen.

FIG. 24 is a diagram showing an example of a list of second tomographic images.

### DETAILED DESCRIPTION

### First Embodiment

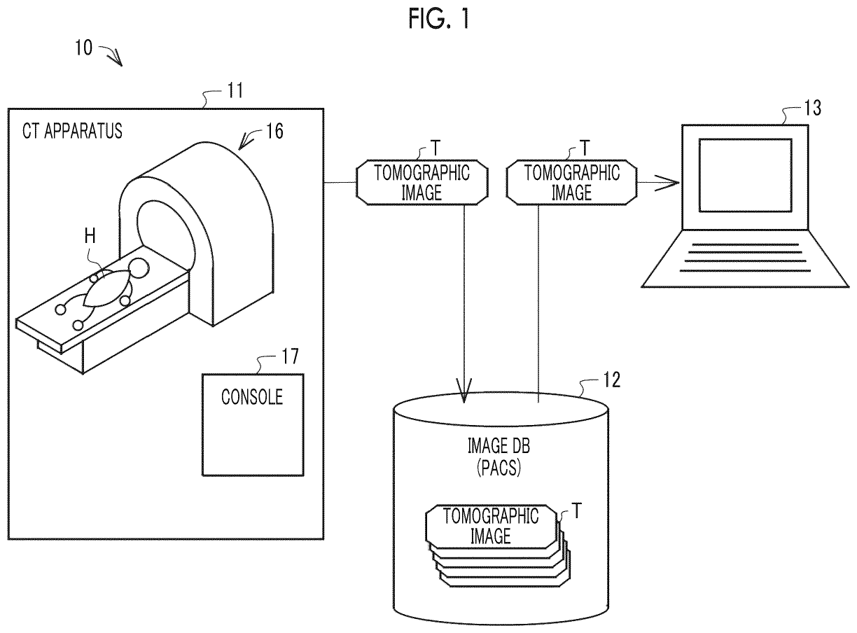

A tomographic imaging system 10 shown in FIG. 1 comprises a CT apparatus 11, an image DB 12, and an image display apparatus 13. The CT apparatus 11 is an example of a PCCT apparatus. The tomographic imaging system 10 is installed in, for example, a medical facility. The image DB 12 is implemented using, for example, a PACS. The image DB 12 stores a tomographic image T of a subject H output from the CT apparatus 11, and delivers the stored tomographic image T to the image display apparatus 13, which is a request source, in response to a request from the image display apparatus 13. The image display apparatus 13 is disposed in, for example, each medical department in the medical facility and is used by a doctor in the medical department.

As is well known, the CT apparatus 11 obtains the tomographic image T of the subject H by imaging the subject H using X-rays as an example of radiation. The CT apparatus 11 is installed in, for example, an imaging room of a radiology department in the medical facility. The CT apparatus 11 comprises a stand 16 and a console 17. The console 17 functions as an operation terminal and a control device for operating the stand 16. The console 17 is operated by an operator such as a radiologic technologist. Further, the console 17 also functions as an image processing apparatus that performs image processing on data output from the stand 16 to generate the tomographic image T.

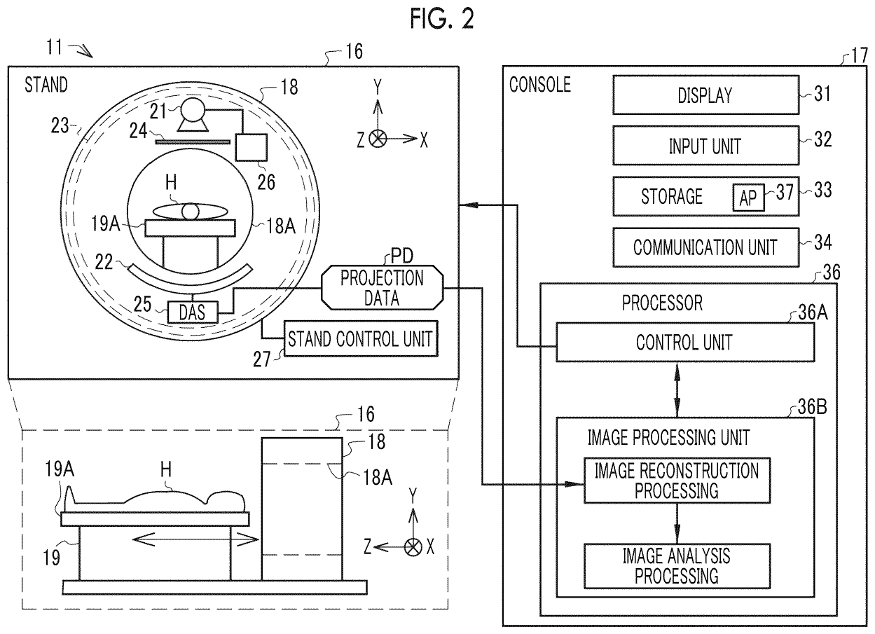

As shown in FIG. 2, the stand 16 is a main unit of the CT apparatus 11, and comprises a gantry 18 and an examination table device 19. In FIG. 2, in addition to a front view of the stand 16, a side view of the stand 16 is shown in a rectangular dashed line frame. The examination table device 19 includes a top plate 19A on which the subject H can be placed in a decubitus posture. The subject H is placed in a posture in which a body axis of the subject H matches a longitudinal direction of the top plate 19A (Z axis direction of the stand 16). The top plate 19A can move in the Z axis direction in a state of being kept horizontal. The gantry 18 has an annular shape as a whole, and a circular opening portion 18A having a larger diameter than a width of the top plate 19A is formed at the center thereof. During the imaging, the top plate 19A on which the subject His placed is moved in the Z axis direction relative to the gantry 18 to enter the opening portion 18A. The imaging is performed while moving the top plate 19A relative to the gantry 18.

A radiation source 21, a detector 22, and a frame 23 are disposed inside the gantry 18. The radiation source 21 emits the radiation toward the subject H. The detector 22 is a radiation detector that detects the radiation transmitted through the subject H. The radiation transmitted through the subject H is attenuated by interaction (absorption, scattering, and the like of the radiation) with structures such as organs and bones in the body of the subject H. The structures each have an attenuation coefficient for the radiation specific to the structures, and the radiation transmitted through the structure carries information reflecting the physical properties of the structures. The detector 22 detects the radiation in which the physical properties of the structure of the body of the subject H are reflected. The detector 22 has a detection surface on which pixels are two-dimensionally arranged and outputs a detection signal for each pixel. Therefore, it is possible to detect the detection signal of the radiation for each transmission position at which the radiation is transmitted through the structure of the subject H. The detector 22 has a substantially arc shape in accordance with a curvature of the gantry 18, and the detection surface is also curved. As will be described later, the detector 22 is a photon-counting detector, and is an example of a “photon-counting radiation detector” according to the disclosed technology.

The radiation source 21 and the detector 22 are disposed at positions facing each other in the gantry 18 and rotate about the Z axis while maintaining a facing posture. The frame 23 has an annular shape and supports the radiation source 21 and the detector 22 so as to be rotatable. During the imaging, the stand 16 detects projection data PD at a plurality of circumferential positions around the Z axis corresponding to the body axis of the subject H while rotating the radiation source 21 and the detector 22 about the subject H on the top plate 19A, by using the detector 22. During the imaging, the top plate 19A is also moved in the Z axis direction in synchronization with the rotation of the radiation source 21 and the detector 22. As a result, the projection data PD of the radiation at each position around the body axis of the subject His acquired.

A data acquisition system (DAS) 25 collects the detection signals output by the detector 22, generates the projection data PD at each position around the Z axis based on the collected detection signals, and outputs the generated projection data PD to the console 17.

On the front side in an irradiation direction of the radiation source 21, an irradiation field limiter 24 (also called a collimator) that limits an irradiation field of radiation is provided. The irradiation field limiter 24 has an irradiation opening of which a contour is defined by a plurality of shielding plates for shielding the radiation, and can change a size of the irradiation opening by moving the shielding plates. Reference numeral 26 denotes a high-voltage generator that generates a high voltage supplied to the radiation source 21. The radiation source 21 and the detector 22 are electrically connected to the frame 23 via a slip ring, and, for example, power supply, transmission and reception of data, and the like are performed via the slip ring. The slip ring connection allows the radiation source 21 and the detector 22 to perform helical scan imaging in which imaging is performed while rotating in one direction without reversing the rotation direction.

The stand 16 is provided with a stand control unit 27. The stand control unit 27 performs control of the respective units of the stand 16 in addition to the rotation of the radiation source 21 and the detector 22 and the movement of the top plate 19A based on an instruction from the console 17.

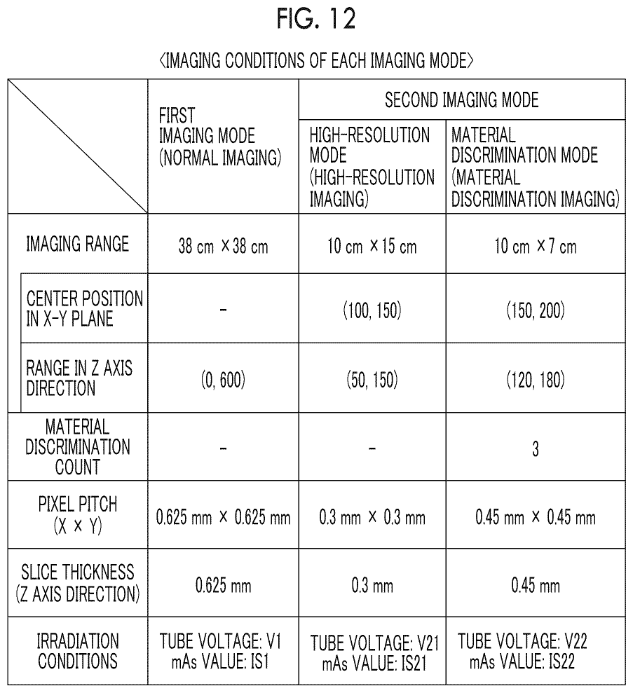

The imaging conditions of the CT apparatus 11 are set through the stand control unit 27 in accordance with the operation from the console 17. The imaging conditions include radiation irradiation conditions of the radiation source 21, an imaging range, a slice thickness, and the like (see FIG. 12). The irradiation conditions of the radiation include a tube voltage (unit: kV) applied to the radiation source 21, a tube current (unit: mA), and an irradiation time (unit: msec) of the radiation. The product of the tube current and the irradiation time defines a total radiation irradiation amount, and is called an mAs value.

The imaging range includes an imaging range in an X-Y plane and an imaging range in the Z axis direction. The imaging range in the Z axis direction is a range in the body axis direction of the subject H, and is determined in accordance with the imaging part, such as the entire body from the vertex to the toe, the head or the abdomen only, or the chest and the abdomen. The imaging range in the Z axis direction is adjusted by changing a movement range of the top plate 19A.

Further, the imaging range in the X-Y plane is a range of a target part to be reconstructed as the tomographic image T representing an axial cross section (corresponding to the X-Y plane) orthogonal to the body axis of the subject H. The imaging range in the X-Y plane corresponds to a so-called field of view (FOV). In a case in which the number of pixels of the detector 22 is the same, the resolution of the X-Y plane is higher as the FOV is smaller, and the resolution of the X-Y plane is lower as the FOV is larger. For example, the resolution is higher in a case in which a smaller region that is a part of the chest is set as the FOV than in a case in which the entire chest of the subject H is set as the FOV.

The FOV is defined by, for example, the irradiation field of the radiation. The irradiation field is adjusted by changing the size of the irradiation opening of the irradiation field limiter 24. In addition, in a case in which the position of the subject H in the opening portion 18A of the gantry 18 is adjusted, a relative positional relationship between the subject H and the radiation source 21 and the detector 22 is adjusted, and thus the irradiation field of the radiation transmitted through the subject H can also be adjusted by this adjustment. In addition, by adjusting the position of the subject H in the opening portion 18A, a center position (corresponding to a center position of the X-Y plane in FIG. 12) of the target part in the tomographic image T can also be changed.

In addition, the slice thickness is an imaging slice thickness and is a parameter that is distinguished from an image reconstruction slice thickness that defines a slice thickness during the image reconstruction. The imaging slice thickness is a parameter that determines the resolution in the Z axis direction, and is defined by, for example, a Z axis direction beam width of the radiation at the transmission position at which the radiation is transmitted through the subject H. The resolution is higher as the slice thickness is smaller. Additionally, in a case of a multi-slice CT in which a plurality of pixel rows are provided in the Z axis direction in the detector 22, there are cases in which the slice thickness is defined by, for example, the width of the detector 22 or the width of a pixel row.

In addition, in a case of the helical scan, as the imaging conditions, a pitch factor, which is a ratio of a movement distance of the top plate 19A during one rotation of the radiation source 21 to the slice thickness, and an effective slice thickness is determined by the pitch factor. The pitch factor is also called a helical pitch.

In addition, the CT apparatus 11 can acquire the tomographic images T having different resolution, and can perform standard imaging and high-resolution imaging at higher resolution than the standard imaging. Furthermore, the CT apparatus 11 can perform material discrimination imaging in addition to the high-resolution imaging. The material discrimination imaging is imaging for acquiring a material discrimination image capable of discriminating and visualizing a plurality of materials having different attenuation coefficients of the radiation, as the tomographic image T. The settings of the imaging modes, including a standard imaging mode, a high-resolution mode, and a material discrimination mode, are also included in the imaging conditions. The imaging conditions are also called imaging protocols or the like.

The console 17 comprises a display 31, an input device 32, a storage 33, a communication unit 34, and a processor 36. As an example, the console 17 is configured on the basis of a personal computer, and the hardware configuration thereof is similar to that of a general-purpose computer. The display 31 is, for example, a liquid-crystal display, and displays an operation screen and the captured tomographic image T. The input device 32 is a device for the operator to input an operation instruction, and is configured with a keyboard, a mouse, and the like.

The storage 33 is a data storage that stores various programs such as a control program controlling the respective units of the console 17. The various programs include an application program 37 causing the processor 36 to function as an image processing apparatus using the control device of the CT apparatus 11 and the console 17. Examples of the storage 33 include a hard disk drive (HDD) and a solid state drive (SSD). In addition, the tomographic image T acquired from the stand 16 is temporarily stored in the storage 33. The application program 37 is an example of an “operation program” according to the disclosed technology.

The communication unit 34 is a communication interface for performing communication between the console 17 and each of the CT apparatus 11 and the image DB 12. The communication unit 34 is connected to a network (not shown) such as a local area network (LAN) and/or a wide area network (WAN), and performs transmission control in accordance with a communication protocol defined in various wired or wireless communication standards.

The processor 36 functions as a control unit 36A that controls the respective units of the console 17, and an image processing unit 36B that executes various types of image processing. The processor 36 is configured with, for example, a central processing unit (CPU) and a memory such as a random access memory (RAM). The CPU functions as the processor 36 by loading the various programs including the application program 37 from the storage 33 into the memory and executing the loaded programs.

The control unit 36A controls the stand 16 through the stand control unit 27 in accordance with the instruction of the operator input from the input device 32. The settings of the imaging conditions and the like are performed by the control unit 36A.

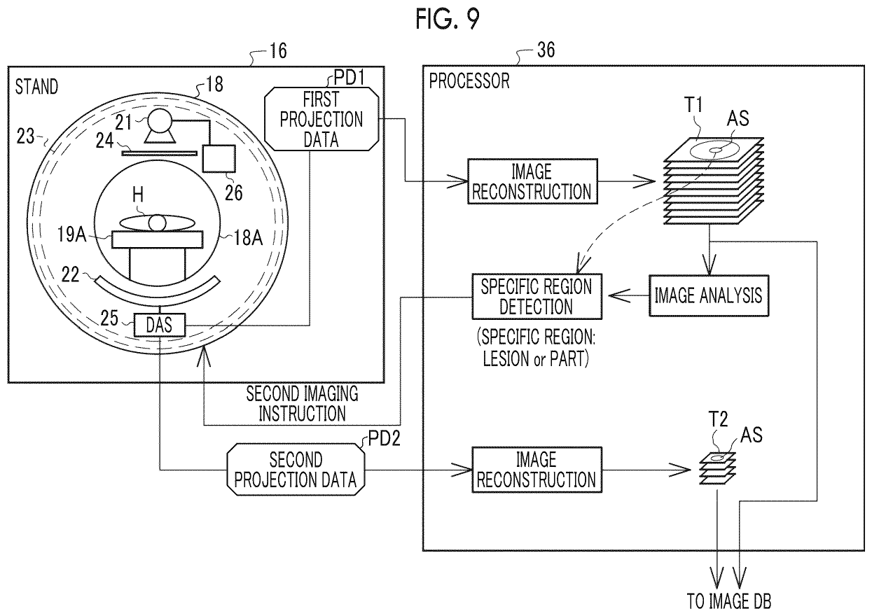

The image processing unit 36B executes image reconstruction processing and image analysis processing. The image reconstruction processing is processing of generating the tomographic image T by reconstructing the tomographic image T based on the projection data PD acquired from the stand 16. The reconstruction of the tomographic image T based on the projection data PD is performed by, for example, a filtered back projection (FBP) method or an iterative reconstruction (IR) method. The image analysis processing is processing of detecting a specific region AS (see FIG. 9 and the like) in the tomographic image T by performing the image analysis on the tomographic image T. The specific region AS is a region that meets a predetermined condition. The predetermined condition is, for example, that the specific region AS includes a specific organ such as the liver and the pancreas or includes a specific abnormal part such as a tumor. That is, in the image analysis processing, the specific organ or abnormal part, such as the pancreas, is detected as the specific region AS from the tomographic image T. The processor 36 is an example of a “processor” according to the disclosed technology.

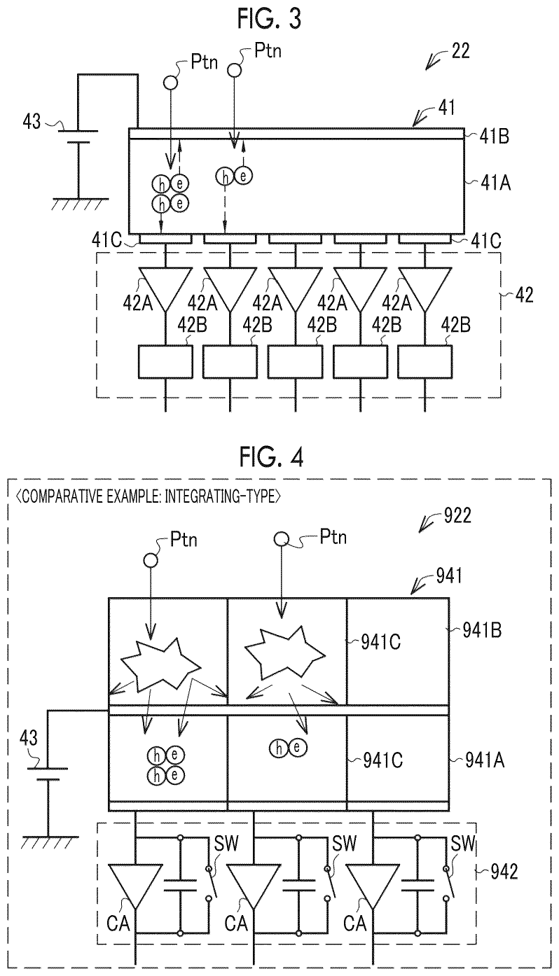

The principle and the function of the detector 22, which is the photon-counting detector, will be schematically described with reference to FIGS. 3 to 8. The photon-counting detector 22 shown in FIG. 3 as an example is a detector that can count the number of photons of incident X-rays. FIG. 4 shows an energy-integrating-type detector 922 in the related art as a comparative example. The detector 22 will be described in comparison with the comparative example as necessary.

In FIG. 3, the detector 22 comprises a panel section 41 and a readout circuit 42. The panel section 41 has a detection surface of the radiation on which pixels that detect the X-rays as the radiation are two-dimensionally arranged. The panel section 41 includes an X-ray conversion layer 41A, a common electrode 41B, and an individual electrode 41C. The X-ray conversion layer 41A is a semiconductor layer that directly converts the incident X-rays into an electric signal. The semiconductor layer forming the X-ray conversion layer 41A is composed of, for example, cadmium telluride (CdTe) or cadmium zinc telluride (CdZnTe). The individual electrode 41C is an electrode corresponding to each pixel, and the common electrode 41B is an electrode common to each pixel. In a case in which photons Ptn of the X-rays are incident on the X-ray conversion layer 41A, the X-ray conversion layer 41A generates a pair of electrons e and holes h as charges in an amount corresponding to the energy of the photons Ptn. Since a bias voltage is applied to the common electrode 41B and the individual electrode 41C from a power supply 43, an electric field is generated in the X-ray conversion layer 41A. Therefore, for example, the electrons e generated in the X-ray conversion layer 41A move to the common electrode 41B, and the holes h move to the individual electrode 41C. As a result, a voltage V corresponding to the charge is generated in each individual electrode 41C. In the X-ray conversion layer 41A, the charge generated for each incident photon Ptn reaches the individual electrode 41C in the order of incidence.

The readout circuit 42 comprises an amplifier 42A and a discriminator 42B for each individual electrode 41C, and reads out the voltage V generated in each individual electrode 41C. In a case in which one photon Ptn is incident, a pulse signal having a voltage value whose magnitude corresponds to the energy of the one photon Ptn is generated in the individual electrode 41C. The amplifier 42A amplifies the pulse signal. The discriminator 42B includes a counter, and counts the number of pulse signals having magnitude equal to or greater than a predetermined threshold value. In the individual electrode 41C, the pulse signal for each incident photon Ptn is sequentially generated, and the pulse signal is amplified by the amplifier 42A and input to the discriminator 42B. The discriminator 42B counts the sequentially input pulse signals.

The readout circuit 42 outputs the number of pulse signals counted for each individual electrode 41C, as the detection signal for each pixel to the DAS 25. The number of pulse signals counted for each individual electrode 41C corresponds to the number of photons Ptn and, as a result, corresponds to an amount of X-rays incident on each pixel. The DAS 25 generates the projection data PD based on the detection signal for each pixel input from the readout circuit 42. As described above, a difference between the photon-counting detector 22 and the integrating-type detector 922 according to the comparative example shown in FIG. 4 is that the photon-counting detector 22 can accurately count the number of photons Ptn of the X-rays incident on each pixel.

The detector 922 shown in FIG. 4 comprises a panel section 941 and a readout circuit 942. The panel section 941 is an indirect conversion type that converts the X-rays into visible light and then converts the visible light into the electric signal. The panel section 941 comprises a scintillator 941B that converts the X-rays into the visible light and a photoelectric conversion layer 941A that converts the visible light into the electric signal. The photoelectric conversion layer 941A has a configuration in which a plurality of photoelectric conversion elements that convert the visible light into the electric signal, such as photodiodes, are two-dimensionally arranged. Each photoelectric conversion element corresponds to the pixel. The scintillator 941B and the photoelectric conversion layer 941A are provided with a partition wall 941C for determining the pixel.

In a case in which the photons Ptn of the X-rays are incident on the scintillator 941B, the visible light is generated, and the generated visible light is diffused in the scintillator 941B. A direction of diffusion is omnidirectional, and a part of the diffused visible light is mixed with visible light generated by subsequent photons Ptn. The diffusion light that is diffused in this way is incident on the photoelectric conversion layer 941A. The photoelectric conversion layer 941A generates a charge corresponding to an amount of the incident diffusion light. The readout circuit 942 reads the charge generated in each pixel of the photoelectric conversion layer 941A. The readout circuit 942 includes, for each pixel, an integrating amplifier CA configured with an amplifier and a capacitor and a reset switch SW. The integrating amplifier CA accumulates the charge generated by the diffusion light incident on each pixel in the photoelectric conversion layer 941A in the capacitor, and outputs the voltage corresponding to the accumulated charge. The integrating amplifier CA accumulates the charge for a certain time, and the voltage corresponding to the accumulated charge is read out as the detection signal. After the readout is performed, the accumulated charge is reset by the reset switch SW.

As described above, in the integrating-type detector 922, the incident photons Ptn become the diffusion light, and the charge generated by the diffusion light is integrated for a certain time and then read out. Therefore, in the detector 922, the detection signal for each pixel is a value obtained by integrating the energies of a plurality of photons Ptn, and the detection signal cannot represent the energy of each photon Ptn individually.

Due to a difference in configuration and principle, the photon-counting detector 22 can achieve, first, higher resolution than the integrating-type detector in the related art and, second, a material discrimination function. First, in order to achieve high resolution, as shown in FIG. 4, the integrating-type detector 922 requires the partition wall 941C to prevent crosstalk between adjacent pixels. Therefore, in the integrating-type detector 922, the partition wall 941C restricts the reduction of a pixel size and a pixel pitch. On the other hand, in the detector 22 shown in FIG. 3, the partition wall 941C is not required, so that the pixel size and the pixel pitch can be further reduced. As a result, the detector 22 can achieve higher resolution than in the related art.

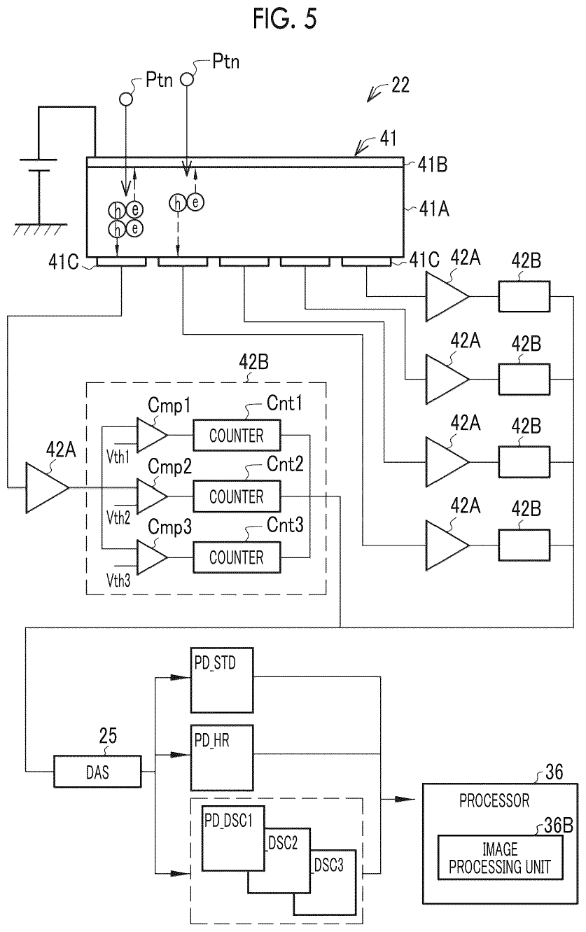

The material discrimination function will be described using FIGS. 5 to 8. More specifically, the discriminator 42B of the detector 22 is configured as shown in FIG. 5 as an example. That is, the discriminator 42B includes a plurality of comparators Cmp1 to Cmp3 and a plurality of counters Cnt1 to Cnt3. Each of the comparators Cmp1 to Cmp3 outputs the pulse signal having the voltage value greater than predetermined threshold voltages Vth1 to Vth3. Each of the counters Cnt1 to Cnt3 connected downstream of the comparators Cmp1 to Cmp3 counts the pulse signal output from each of the comparators Cmp1 to Cmp3.

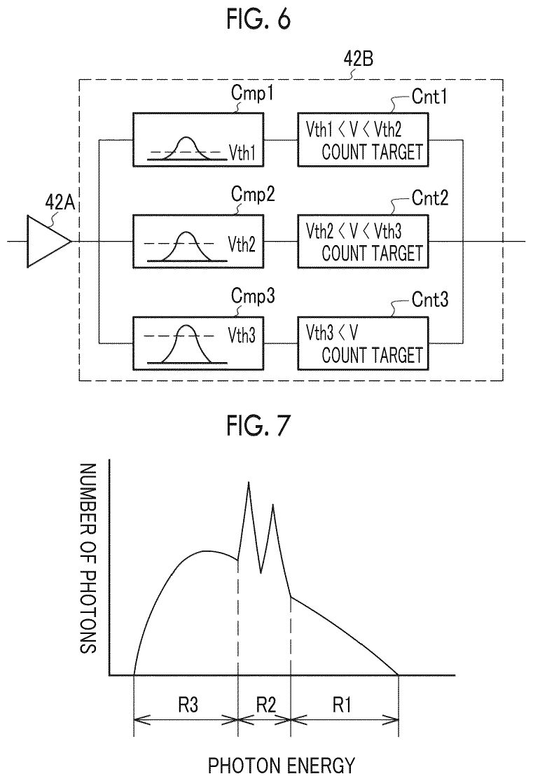

FIG. 6 schematically shows the function of the discriminator 42B, as an example. As shown in FIG. 6, for example, the threshold voltage Vth1 of the comparator Cmp1 is the minimum, the threshold voltage Vth3 of the comparator Cmp3 is the maximum, and the threshold voltage Vth2 of the comparator Cmp2 is set to be between the threshold voltages Vth1 and Vth3. The counter Cnt3 counts the pulse signal that exceeds the maximum threshold voltage Vth3. The counter Cnt2 counts the pulse signal that exceeds the intermediate threshold voltage Vth2. By subtracting the number of pulse signals counted by the counter Cnt3 from the number counted by the counter Cnt2, the number of pulse signals having the voltage value greater than the threshold voltage Vth2 and smaller than the threshold voltage Vth3 can be counted. Although this subtraction processing is shown as being performed by the counter Cnt2, the subtraction processing may be performed by another operation circuit. Similarly, the counter Cnt1 can count the number of pulse signals having the voltage value smaller than the threshold voltage Vth2 by counting the pulse signal greater than the threshold voltage Vth1 and subtracting the count number counted by the counter Cnt2 from the count number of the pulse signals having the voltage value greater than the threshold voltage Vth1.

As shown in FIG. 7 as an example, the X-rays have an energy distribution, which indicates that the emitted X-rays include the photons Ptn having different energies. As shown in FIG. 7, in general, the energy spectrum of the X-rays includes bremsstrahlung, represented by a continuous curve, and characteristic X-rays that appear as peaks in certain energy bands. For example, by making the ranges R1 to R3 of the photon energy as shown in FIG. 7 to correspond to the magnitude of the voltages of the pulse signals counted by the counters Cnt1 to Cnt3 of the discriminator 42B, the X-ray energy spectrum can be acquired in a separated form. The energy band of each of the ranges R1 to R3 is called a bin or the like. In FIG. 7, since the energy band is separated into three bands of high, medium, and low, the method is called a three-bin method or the like.

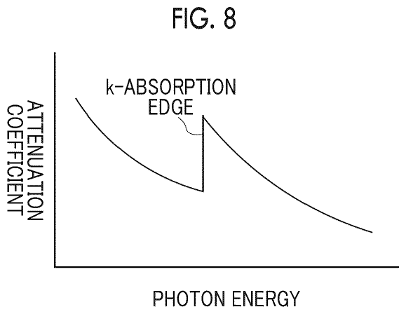

As shown in FIG. 8 as an example, the attenuation coefficient of the X-rays in the material generally tends to decrease as the photon energy increases, but it is known that there are significant points such as a k-absorption edge that show high absorption at some photon energies. The photon energy absorbed by the k-absorption edge shows a value specific to the material. Therefore, by appropriately setting the plurality of threshold voltages Vth of the discriminator 42B, such as the threshold voltages Vth1 to Vth3, in accordance with the k-absorption edge of the material to be discriminated, the material can be discriminated and visualized, for example, as an abnormal part including a lesion and a normal part, or different plurality of organs. The material discrimination image in which a specific material is emphasized is obtained, for example, in the same manner as the energy subtraction image, by generating the tomographic images corresponding to the plurality of energy bands and subtracting the tomographic images from each other.

As shown in FIG. 5, the DAS 25 generates the projection data PD based on the pulse signals output from the discriminator 42B. The DAS 25 generates, for example, projection data PD_STD for standard resolution as the projection data PD in a case of generating the tomographic image T having standard resolution as the tomographic image T. In a case of generating the tomographic image T having high resolution, the DAS 25 generates the projection data PD_HR for high resolution as the projection data PD. In a case of generating the material discrimination image in which a plurality of materials having different attenuation coefficients of radiation are discriminated and visualized as the tomographic image T, the DAS 25 generates projection data PD_DSC1, PD_DSC2, and PD_DSC3 for material discrimination, which can be reconstructed into the material discrimination image. Each of the projection data PD_DSC1, PD_DSC2, and PD_DSC3 is the projection data PD in which the information on the material having the different attenuation coefficient is emphasized.

In modes other than the material discrimination mode, the DAS 25 can generate the projection data PD based on the total number of photons Ptn incident on each pixel by accumulating the count numbers output from the counters Cnt1 to Cnt3 of the discriminator 42B. In a case of changing the resolution, for example, the resolution can be lowered by summing counts of the pulse signals output from the discriminator 42B of each pixel across adjacent pixels. As a result, the projection data PD_HR for high resolution and the projection data PD_STD for standard resolution can be generated.

In the present example, the example has been described in which the discriminator 42B can set three threshold voltages Vth1 to Vth3, but the discriminator 42B may set two threshold voltages or four or more threshold voltages. The number of materials that can be discriminated also changes in accordance with the number of threshold voltages Vth.

In a case of performing the high-resolution imaging and the material discrimination imaging using the photon-counting detector 22, the amount of data of the generated tomographic image T is significantly larger than that in standard-resolution imaging. In the high-resolution imaging, the number of pixels increases with the increase in resolution, and thus the amount of data increases. In the material discrimination imaging, the number of images also increases in accordance with the number of discriminated materials even in a case in which the resolution is standard, and thus the amount of data increases. In a case of combining the material discrimination imaging and the high-resolution imaging, the amount of data further increases. Therefore, in a case of using the photon-counting CT apparatus 11, there is a concern that the capacity of the image DB 12 is strained.

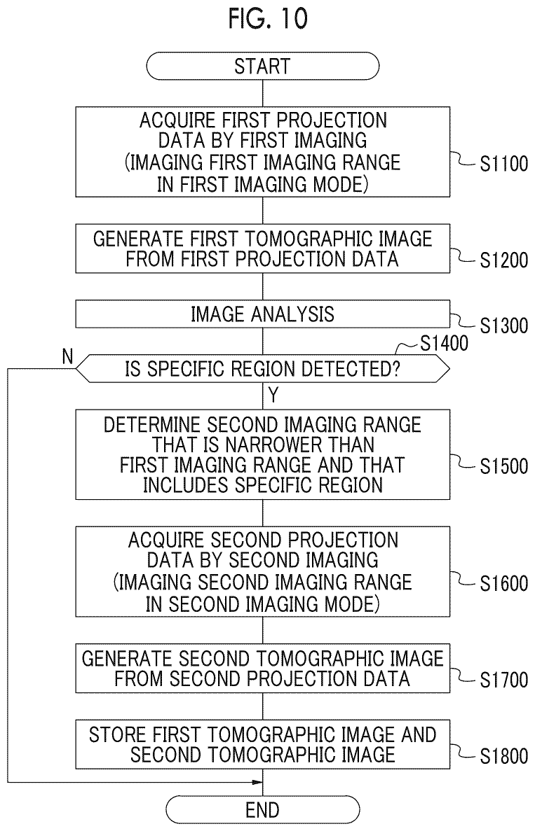

Therefore, the CT apparatus 11 executes processing as shown in FIGS. 9 and 10 as an example. As shown in FIG. 10, first, in step S1100, the CT apparatus 11 performs first imaging in which first projection data PD1 is acquired by imaging a first imaging range in a first imaging mode. The first imaging mode is, for example, a standard-resolution imaging mode, and the first imaging is also referred to as normal imaging.

Next, in step S1200, the processor 36 reconstructs the first projection data PD1 to generate a first tomographic image T1. In step S1300, the processor 36 detects the specific region AS that meets the predetermined condition, such as the specific organ or the abnormal part including the lesion, by performing the image analysis on the first tomographic image T1.

In a case in which the specific region AS is not detected in step S1400 (N in step S1400), the processor 36 ends the processing. On the other hand, in a case in which the specific region AS is detected (Y in step S1400), the processing proceeds to step S1500. In step S1500, the processor 36 determines a second imaging range that is narrower than the first imaging range, which is the imaging range of the first tomographic image T1, and that includes the specific region AS.

In step S1600, the processor 36 executes second imaging in which second projection data PD2 is acquired by imaging a second imaging range in a second imaging mode. The second imaging is, for example, imaging in the high-resolution mode or the material discrimination mode. In step S1700, the processor 36 reconstructs the second projection data PD2 acquired by the second imaging to generate a second tomographic image T2. In step S1800, the processor 36 stores the data of the first tomographic image T1 and the data of the second tomographic image T2 in the image DB 12. The first tomographic image T1 and the second tomographic image T2 are read out by the image display apparatus 13 and used for diagnosis.

Since the second imaging range of the second tomographic image T2 is a part of the first imaging range, the second imaging range of the second tomographic image T2 is narrower than the first imaging range of the first tomographic image T1. Therefore, the amount of data stored in the image DB 12 can be reduced as compared with a case in which the same imaging range as that in the first imaging is imaged in the second imaging, and both the tomographic images of the first imaging and the second imaging are stored in the image DB 12. As a result, it is possible to utilize the high-resolution image or the material discrimination image that can be acquired by the photon-counting radiation detector while suppressing the amount of data to be stored.

In particular, in a case in which the second imaging is performed in the high-resolution mode or the material discrimination mode, the amount of data of the second tomographic image T2 is significantly increased. The disclosed technology is particularly effective in a case of the photon-counting CT apparatus 11 that can perform such imaging. In addition, the amount of data also affects the data transfer speed and transfer time between the CT apparatus 11 and the console 17. In a case in which the data transfer speed of the communication unit 34 is the same as that in the related art, and the amount of data is large, a delay may occur in the transfer of the projection data PD. The delay in the data transfer of the projection data PD can be suppressed by narrowing the imaging range of the second tomographic image T2.

In addition, since the imaging range is narrowed in the second imaging, the radiation exposure to the subject H is also suppressed.

The processor 36 generates the first tomographic image T1 by reconstructing the first projection data PD1 obtained by performing imaging in the first imaging mode, and generates the second tomographic image T2 by reconstructing the second projection data PD2 obtained by performing the second imaging in the second imaging mode. As described above, the processor 36 acquires the first projection data PD1 for generating the first tomographic image T1 and the second projection data PD2 for generating the second tomographic image T2 by performing imaging for each of the first projection data PD1 and the second projection data PD2. Therefore, it is possible to set appropriate imaging conditions for each imaging.

By performing the second imaging, it is also possible to set the irradiation conditions different from the first imaging as the imaging conditions. In addition, the imaging conditions include adjusting the threshold voltage Vth of the discriminator 42B in the material discrimination mode. By adjusting the threshold voltage Vth, information on an appropriate energy band corresponding to the material to be discriminated in the second tomographic image T2 can be acquired by separating the information from information on the other energy bands. As described above, the imaging conditions can be appropriately set by performing the second imaging for obtaining the second tomographic image T2.

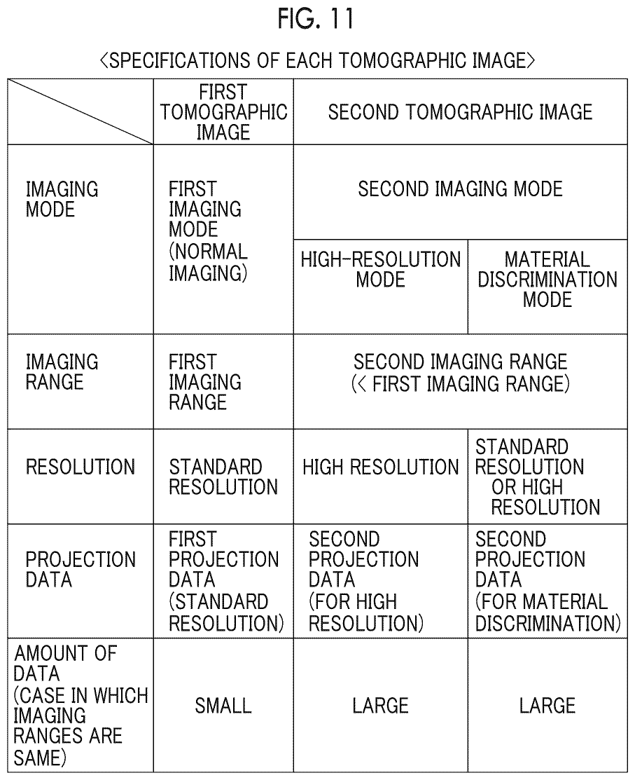

The specifications of the first tomographic image T1 and the second tomographic image T2 are summarized as shown in FIG. 11 as an example. The first imaging mode of the first tomographic image T1 is the normal imaging mode, and the second imaging mode of the second tomographic image T2 is the high-resolution mode or the material discrimination mode. The second imaging range of the second tomographic image T2 is narrower than the first imaging range. The first tomographic image T1 has standard resolution, whereas the second tomographic image T2 has high resolution in the high-resolution mode and has standard resolution or high resolution in the material discrimination mode. The first projection data PD1 of the first tomographic image T1 is the projection data PD_STD for standard resolution. The second projection data PD2 of the second tomographic image T2 is the projection data PD_HR for high resolution, as shown in FIG. 5, in a case in which the second tomographic image T2 is the high-resolution image. In addition, in a case in which the second tomographic image T2 is the material discrimination image, the second projection data PD2 is the projection data PD_DSC1 to PD_DSC3 for material discrimination, as shown in FIG. 5. In a case in which the comparison is performed in the same imaging range, the amount of data of the second tomographic image T2 is larger than the amount of data of the first tomographic image T1.

In addition, FIG. 12 shows an example of the imaging conditions in the first imaging mode and the second imaging mode. In the present example, the first imaging range of the first imaging mode has a FOV that is the imaging range of the X-Y plane and is 38 cm×38 cm. On the other hand, in a case in which the second imaging mode is the high-resolution mode, the FOV of the second imaging range is 10 cm×15 cm, and in a case in which the second imaging mode is the material discrimination mode, the FOV of the second imaging range is 10 cm×7 cm. In addition, it is possible to set coordinates of the center position of the X-Y plane as the imaging conditions. In the example shown in FIG. 12, for example, a range in which an entire region around the body axis of the subject H is included is set as the first imaging range. In FIG. 12, the center position of the first imaging range is omitted. Meanwhile, since the second imaging range is a part of the first imaging range, the center position is set at any position within the range with the first imaging range as the maximum region, and the FOV with the set center position as the base point is set. In addition, in a case in which the range of the first imaging mode is set as the maximum region, in the second imaging mode, a part thereof is set as the range in the Z axis direction.

In the material discrimination mode, the number of materials to be discriminated can be set. In the present example, “3” is set. Although not shown in FIG. 12, in the material discrimination mode, it is possible to set the threshold voltage Vth corresponding to the material to be discriminated.

In addition, a small value is set for the pixel pitch in the high-resolution mode and the material discrimination mode as compared with the first imaging mode of the normal imaging. Further, the slice pitch is set in accordance with the pixel pitch, for example, a smaller value is set for the slice pitch as the pixel pitch is smaller.

Furthermore, different values are set for the irradiation conditions in each imaging mode.

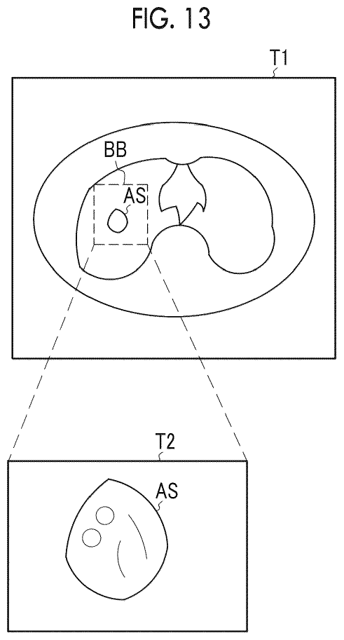

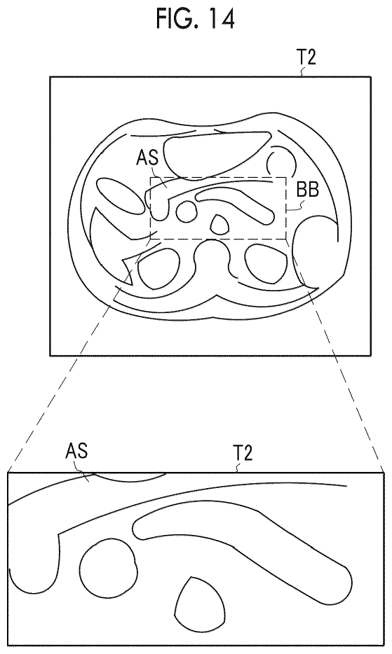

FIGS. 13 and 14 show specific examples of the first tomographic image T1 and the second tomographic image T2. In the example shown in FIG. 13, the first tomographic image T1 is an image obtained by setting the entire chest or abdomen as the first imaging range and imaging the set first imaging range at standard resolution. The second tomographic image T2 is an image obtained by setting a part of the specific region AS including the tumor in the chest or the abdomen as the second imaging range and imaging the set second imaging range at high resolution.

In addition, in the example shown in FIG. 14, the first tomographic image T1 is an image obtained by setting the entire abdomen as the first imaging range and imaging the set first imaging range at standard resolution. The second tomographic image T2 is an example of an image obtained by setting a part of the specific region AS including the pancreas in the abdomen as the second imaging range and imaging the set second imaging range at high resolution.

In the example shown in FIGS. 13 and 14, the specific region AS in the first tomographic image T1 is surrounded by a bounding box BB. A region corresponding to the bounding box BB is the second imaging range of the second tomographic image T2. The specific region AS is preferably a three-dimensional region. According to the disclosed technology, it is possible to observe the specific region AS in detail with a clear image having high resolution while suppressing the amount of data to be stored.

A clinical significance of such processing is, for example, as follows. For example, in surgery for lung cancer or rectal cancer, it is very important to accurately ascertain a region of the cancer to be resected, including the depth of the cancer, in order to minimize the resection region. By acquiring the second tomographic image T2 as the high-resolution image or the material discrimination image, it is possible to accurately ascertain the resection region of the cancer as compared to the related art. In addition, in a case in which the specific region AS is three-dimensionally ascertained, it is possible to more accurately understand the depth of the cancer.

In addition, pancreatic cancer has the lowest five-year survival rate among all organs, and is a tumor for which early detection is extremely important. Nevertheless, the pancreatic cancer is very small and relatively difficult to detect at an early stage. Accordingly, in order to achieve early detection, it is extremely important to detect abnormalities indicative of pancreatic cancer, such as pancreatic atrophy, dilation of the pancreatic duct, and pancreatic duct obstruction, before the pancreatic cancer is visible on imaging. Therefore, the high-resolution image and, as necessary, the material discrimination image are acquired as the second tomographic image T2. The high-resolution image improves visibility of a small pancreatic duct of about 1.2 mm. Further, by discriminating and visualizing the pancreatic duct portion and the pancreatic parenchyma with the material discrimination image, it is easy to identify both the pancreatic duct portion and the pancreatic parenchyma. In addition, the high-resolution image and the material discrimination image make it easier to recognize the complex morphology of the pancreas and are expected to enable morphological diagnoses such as atrophy of the pancreatic tail and atrophy of the pancreatic body.

In the above-described example, the processor 36 detects the specific region AS from the first tomographic image T1 and determines the second imaging range, which is an example of the imaging conditions, based on the detected specific region AS. Since the processor 36 determines the imaging conditions such as the imaging range, the convenience of a user is improved. In addition, the processor 36 may determine the imaging condition other than the imaging range, such as the resolution, as the imaging conditions of the second imaging. In addition, the processor 36 may determine the irradiation conditions in a case in which appropriate irradiation conditions corresponding to the specific region AS can be determined based on the specific region AS.

Further, the processor 36 may determine the imaging mode as the imaging conditions based on the specific region AS. For example, in a case in which a lung cancer candidate is detected, it is meaningful to clinically confirm the detailed morphological information. Therefore, in a case in which the lung cancer candidate is detected as the specific region AS, the processor 36 selects the high-resolution mode as the second imaging mode. Alternatively, in a case in which a liver cancer candidate is detected, it is meaningful to clinically confirm a qualitative state of the cancer. Therefore, in a case in which the liver cancer candidate is detected as the specific region AS, the processor 36 selects the material discrimination mode as the second imaging mode. Further, in a case in which the rectal cancer is detected, the high-resolution image is highly necessary in order to confirm the extent of cancer invasion into the intestine. In addition, the material discrimination image is highly necessary to clearly ascertain a boundary between the cancer and the normal tissue. Therefore, in a case in which the rectal cancer is detected, the processor 36 selects both the high-resolution mode and the material discrimination mode as the second imaging mode. As described above, the processor 36 may determine the imaging mode of the second imaging in accordance with the structure included in the specific region AS.

In addition, the specific region AS is the region that meets the predetermined condition, and the predetermined condition is that the specific region AS includes the specific organ or the abnormal part. Since the specific organ or the abnormal part is often a region of interest of the doctor in diagnosis, it is effective to extract such a specific region AS.

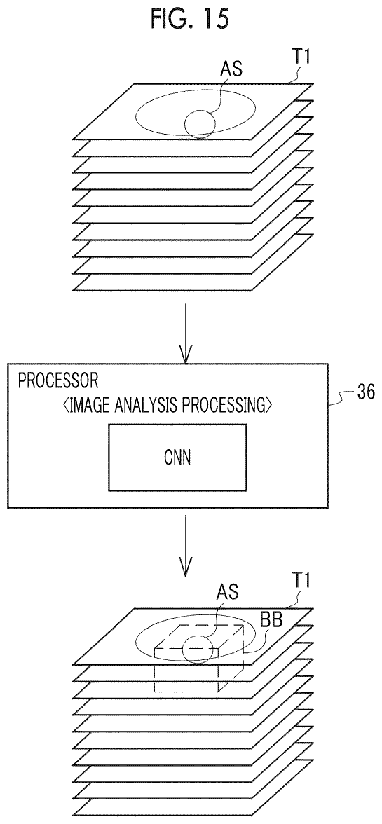

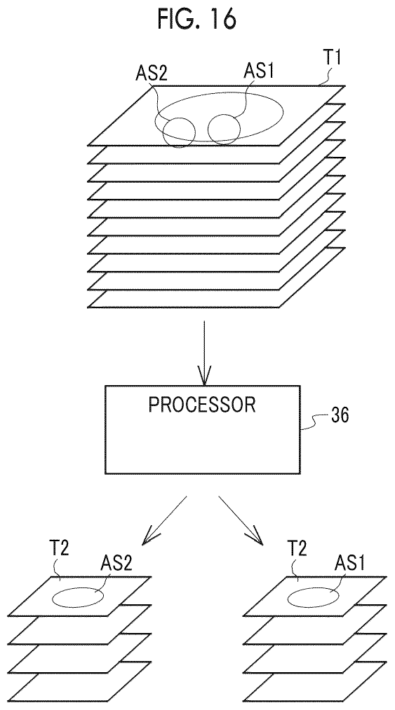

In addition, as shown in FIG. 15, the processor 36 may perform the image analysis for detecting the specific region AS from the first tomographic image T1, using a machine learning model. As the machine learning model, as an example, a convolutional neural network (CNN) is used. As is well known, the CNN includes an encoder that repeats, in stages, processing of applying a plurality of types of filters to an input image to extract a multi-dimensional feature map and image reduction processing by pooling, to extract various features from global features of the input image to fine features. The CNN performs object recognition in the input image based on the extracted feature. By training the CNN in advance with the abnormal part such as the lesion or the specific organ as a recognition target, the CNN can extract a region including the specific region AS such as the abnormal part or the specific organ as indicated by the bounding box BB in FIG. 15. In addition, by detecting the specific region AS for each of a plurality of first tomographic images T1 that are continuous in the body axis direction, the specific region AS can be detected as a three-dimensional region as shown in FIG. 15.

It may be possible to accurately detect the specific region AS by using the machine learning model. The image analysis may be performed by a rule-based method such as pattern matching in addition to the method using the machine learning model.

In addition, as an example, as shown in FIG. 16, in a case in which a plurality of specific regions AS are detected in the first tomographic image T1, the processor 36 may generate the second tomographic image T2 for each of specific regions AS1 and AS2. In this way, the second tomographic image T2 can be observed for each of the plurality of specific regions AS1 and AS2.

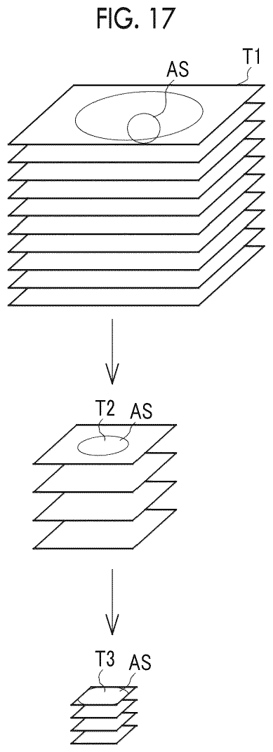

In addition, as an example, as shown in FIG. 17, the processor 36 may further perform the image analysis on the second tomographic image T2 and generate a third tomographic image T3 of a third imaging range that is narrower than the second imaging range of the second tomographic image T2 based on the result of the image analysis. For example, the processor 36 acquires the first tomographic image T1 having standard resolution, and then acquires the second tomographic image T2 having high resolution. Then, the processor 36 determines whether or not the third tomographic image T3 is necessary, by performing the image analysis on the second tomographic image T2. For example, in a case in which the processor 36 determines that the material discrimination image is necessary as the third tomographic image T3, the processor 36 selects to perform third imaging in the material discrimination mode for the third imaging range that is narrower than the second imaging range of the second tomographic image T2. As a result, the processor 36 acquires the third tomographic image T3. More detailed diagnosis can be performed for the specific region AS using the third tomographic image T3.

A clinical specific example of the example in FIG. 17 is as follows. First, in a case in which the prostate region is detected as the specific region AS based on the first tomographic image T1, the processor 36 selects the high-resolution mode as the second imaging mode to detect prostate cancer within the prostate region. Then, in a case in which the prostate cancer is detected as the specific region AS in the second tomographic image T2, there is a high necessity to investigate the possibility of the metastasis of the prostate cancer. Therefore, the material discrimination mode is selected as the third imaging mode in order to acquire the material discrimination image as the third tomographic image T3 such as a spinal region and a liver region. Metastasis diagnosis is performed based on the third tomographic image T3 of the material discrimination image.

As described above, by gradually narrowing the imaging range, it is possible to acquire a detailed image for a necessary portion while determining whether or not the next imaging is necessary based on the tomographic images T generated in stages. As a result, more detailed diagnosis can be performed while suppressing the amount of data to be stored. As a result, the convenience of the user may be improved.

A detection result of the specific region AS based on the first tomographic image T1 and/or a detection result of the specific region AS based on the second tomographic image T2 may be presented to the user as necessary. Then, the detection result may be presented to the user, and the tomographic image T of the specific region AS may be acquired while waiting for an instruction from the user. That is, the processing from the detection of the specific region AS to the acquisition of the next tomographic image T may be performed semi-automatically with the intervention of the instruction from the user, instead of performing the processing in a fully automatic manner.

In the above-described embodiment, the arrangement of the semiconductor layer forming the X-ray conversion layer 41A of the detector 22 may be, for example, arrangement (hereinafter referred to as edge-on arrangement) in which an end surface parallel to the thickness direction of the semiconductor layer is set as the X-ray incidence surface as disclosed in U.S. Pat. No. 8,183,535B. The conventional arrangement in the related art is arrangement in which the surface orthogonal to the thickness direction of the semiconductor layer formed on the substrate serves as the X-ray incidence surface (herein referred to as face-on arrangement); however, in the face-on arrangement, the X-rays are incident along the thickness direction of the semiconductor layer. In a case of the photon-counting type, it is necessary to count each photon Ptn, but in the face-on arrangement, the distance for detecting the photons Ptn depends on the thickness of the semiconductor layer, which has a problem that the distance is too short to be obtained in the thickness. Therefore, by using the edge-on arrangement, the direction orthogonal to the thickness direction is the incidence direction of the photons Ptn, and thus the distance for detecting the photons Ptn can be increased. As a result, it is possible to accurately count the photons Ptn and accurately quantify the photons Ptn.

### Second Embodiment

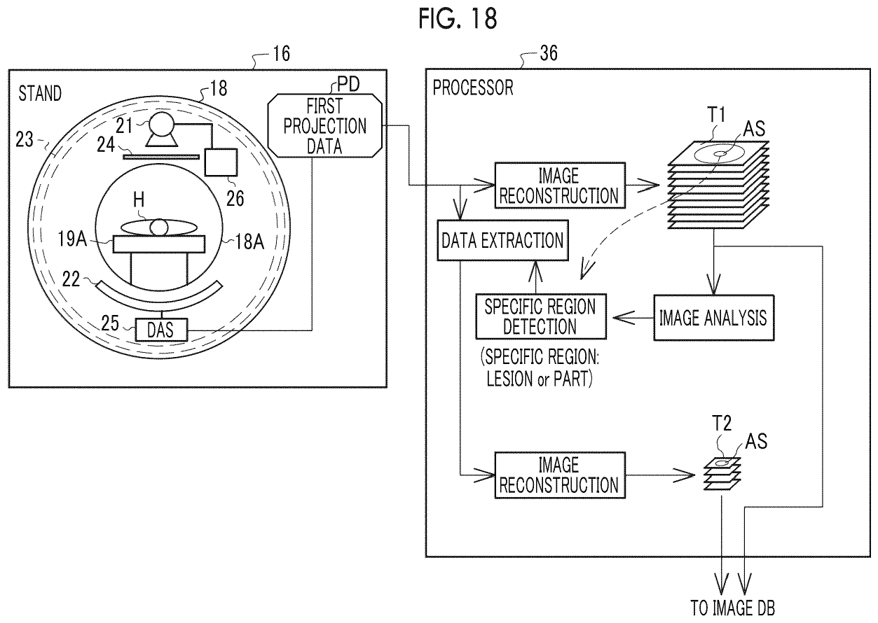

Although, in the first embodiment, the example has been described in which the first tomographic image T1 and the second tomographic image T2 are acquired by performing two imaging operations of the first imaging and the second imaging, as in a second embodiment shown in FIGS. 18 to 20 as an example, the first tomographic image T1 and the second tomographic image T2 may be acquired by performing a single imaging operation.

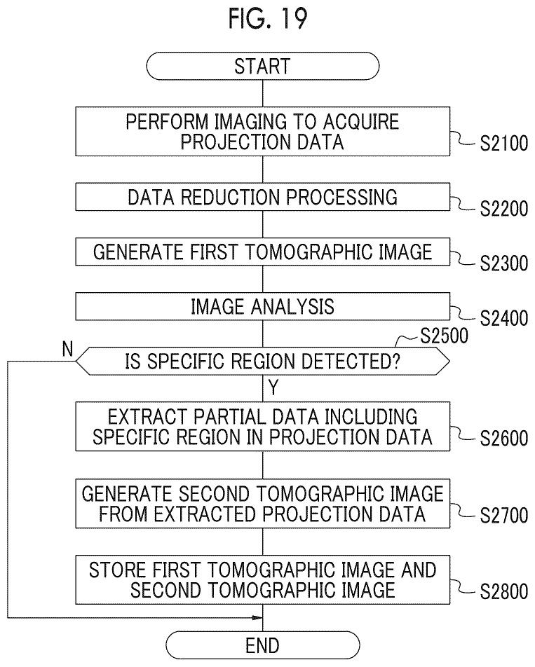

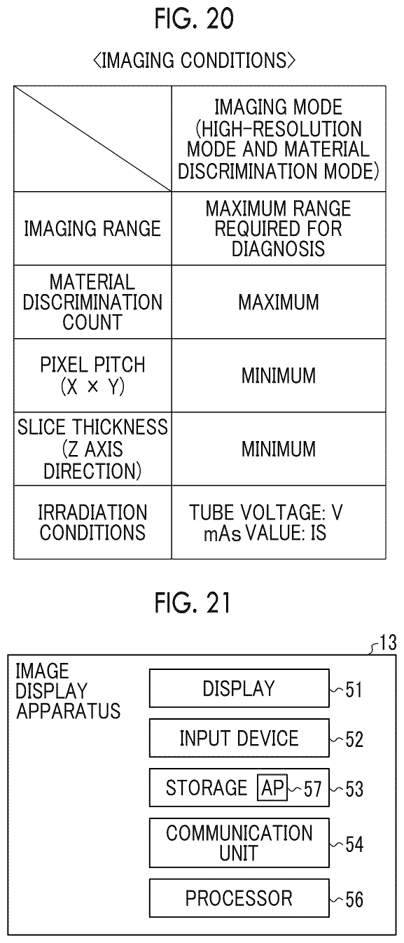

In the second embodiment, as shown in FIGS. 18 and 19, the imaging is performed by the CT apparatus 11 in step S2100. As a result, the projection data PD is acquired. The imaging conditions for this imaging are shown in FIG. 20 as an example. First, the high-resolution mode and the material discrimination mode are provided as the imaging modes, and the imaging range is set to a maximum range required for the diagnosis of the subject H. The number of materials to be discriminated is the maximum. The pixel pitch and the slice thickness are set to be the minimum. The irradiation conditions are set to conditions that can also be applied to the high-resolution mode and the material discrimination mode in accordance with the diagnostic purpose and the imaging part.

Since the resolution of the projection data PD is the maximum, the resolution of the first tomographic image T1 may be too high. Therefore, in step S2200, the processor 36 performs the data reduction processing on the projection data PD and generates the first tomographic image T1 based on the data-reduced projection data PD. The data reduction processing is processing of lowering the resolution, for example, by thinning out pixels or adding pixels.

Next, in step S2300, the processor 36 generates the first tomographic image T1 by reconstructing the acquired projection data PD. In step S2400, the processor 36 detects the specific region AS by performing the image analysis based on the first tomographic image T1. In a case in which the specific region AS is not detected in step S2500 (N in step S2500), the processing ends. On the other hand, in a case in which the specific region AS is detected (Y in step S2500), the processor 36 proceeds to step S2600.

In step S2600, the processor 36 extracts a partial data including the specific region AS in the acquired projection data PD. In step S2700, the processor 36 generates the second tomographic image T2 by reconstructing the extracted projection data PD. In step S2700, the processor 36 generates the second tomographic image T2 without performing the data reduction processing on the projection data PD. In step S2800, the processor 36 stores the generated first tomographic image T1 and second tomographic image T2 in the image DB 12.

In the second embodiment, the second tomographic image T2 is reconstructed by using a part of the projection data PD used for the reconstruction of the first tomographic image T1. Therefore, the second tomographic image T2 can be acquired without performing re-imaging.

However, in the second embodiment, since the projection data PD for generating the first tomographic image T1 and the projection data PD for generating the second tomographic image T2 are the same, the projection data PD having different imaging conditions including the irradiation conditions cannot be used. As described above, in a case in which it is desired to change the imaging conditions for obtaining the projection data PD of each of the first tomographic image T1 and the second tomographic image T2, the first embodiment is preferable.

As in the first embodiment, in the second embodiment, in a case in which the second tomographic image T2 is the high-resolution image, the projection data PD is the projection data PD_HR for high resolution. In a case in which the second tomographic image T2 is the material discrimination image, the projection data PD is the projection data PD_DSC1 to PD_DSC3 for material discrimination. In the above-described example, since the second tomographic image T2 is the high-resolution image and the material discrimination image, the projection data PD is also the projection data PD for high resolution and for material discrimination. Since the high-resolution image or the material discrimination image has a larger amount of data than the normal tomographic image T, it is effective to narrow the imaging range of the second tomographic image T2.

In addition, in the above-described example, the projection data PD is the projection data PD for high resolution and for material discrimination. In a case of generating the first tomographic image T1, the processor 36 performs the data reduction processing on the projection data PD, and then reconstructs the first tomographic image T1 based on the projection data PD on which the data reduction processing has been performed. In a case in which high resolution or the like is not required for the first tomographic image T1, the reconstruction time of the first tomographic image T1 can be shortened by performing the data reduction processing.

### “Image Display Apparatus”

A display example of the first tomographic image T1 and the second tomographic image T2 in the image display apparatus 13 will be described with reference to FIGS. 21 to 23. First, as shown in FIG. 21, the image display apparatus 13 comprises a display 51, an input device 52, a storage 53, a communication unit 54, and a processor 56. The image display apparatus 13 is configured based on a personal computer or the like, similarly to the console 17, and has a hardware configuration that is the same as that of a general-purpose computer.

The display 51 is, for example, a liquid-crystal display, and displays the operation screen, and the first tomographic image T1 and the second tomographic image T2 that are read from the image DB 12. The input device 52 is a device for the operator to input the operation instruction, and is configured with a keyboard, a mouse, and the like.

The storage 53 is a data storage that stores various programs such as a control program that controls the respective units of the image display apparatus 13. The various programs include an application program 57 that is an example of an operation program for causing the computer to function as the image display apparatus 13. Examples of the storage 53 include an HDD and an SSD.

The communication unit 54 is a communication interface for performing communication with the image DB 12. The communication unit 54 is connected to a network (not shown) such as a LAN and/or a WAN, and performs transmission control in accordance with a communication protocol defined in various wired or wireless communication standards.

The processor 56 functions as a control unit that controls the respective units of the image display apparatus 13 and an image processing unit that performs various types of image processing. The processor 56 is configured with a memory, such as a CPU and a RAM, as an example. The CPU functions as the processor 56 by loading the various programs including the application program 57 from the storage 53 into the memory and executing the loaded programs. The processor 56 is an example of a “display control processor that controls display of an image display apparatus” according to the disclosed technology.

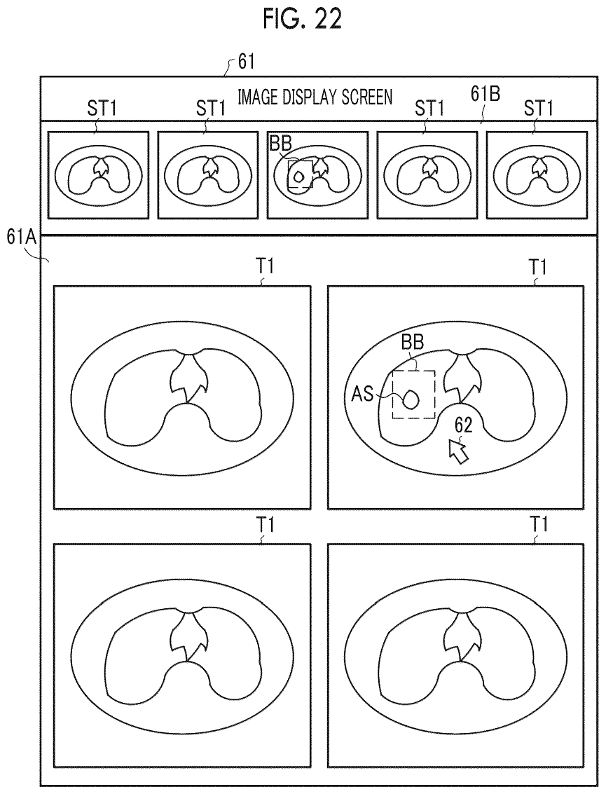

For example, an image display screen 61 as shown in FIG. 22 is displayed on the display 51. The image display screen 61 is provided with a main display region 61A and a sub-display region 61B. The main display region 61A is a region in which the first tomographic image T1 and the like are displayed. The main display region 61A can display a plurality of first tomographic images T1, and for example, four first tomographic images T1 are displayed in square arrangement. The sub-display region 61B is a region in which a thumbnail image ST1, which is a size-reduced image of the first tomographic image T1, is displayed. For example, the thumbnail images ST1 are arranged in the sub-display region 61B in order along the body axis direction. A scroll operation of switching the thumbnail image ST1 to be displayed in the sub-display region 61B can also be performed.

In a case in which a desired thumbnail image ST1 is selected by the scroll operation in the sub-display region 61B, the first tomographic image T1 corresponding to the selected thumbnail image ST1 and the first tomographic image T1 adjacent to the first tomographic image T1 are displayed in the main display region 61A. A scroll operation of switching the first tomographic image T1 to be displayed in the main display region 61A can also be performed.

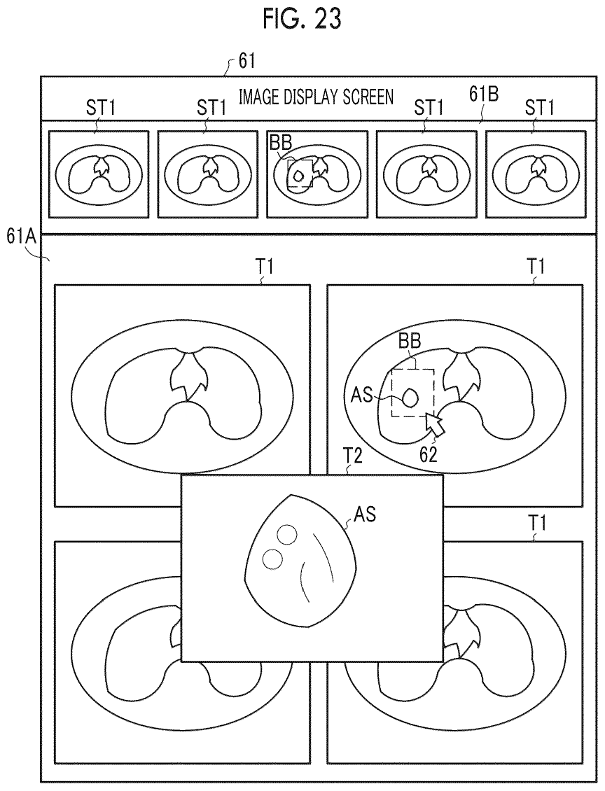

The bounding box BB is displayed on the first tomographic image T1 in which the specific region AS is detected and the thumbnail image ST1 corresponding to the first tomographic image T1. In a case in which the bounding box BB is designated by a mouse pointer 62 and the click operation is performed, the second tomographic image T2 corresponding to the specific region AS is displayed as shown in FIG. 23. The second tomographic image T2 is the high-resolution image or the material discrimination image. The second tomographic image T2 is displayed in a pop-up form so as to overlap a part of the first tomographic image T1 as an example. In this way, the specific region AS corresponds to the second imaging range of the second tomographic image T2. The bounding box BB is an example of an “indicator” according to the disclosed technology.

In this manner, in a case in which the first tomographic image T1 is displayed on the display 51, the processor 56 displays the bounding box BB indicating the specific region AS, which is the second imaging range, in the first tomographic image T1. In a case in which the designation operation is performed on the bounding box BB, the processor 56 displays the second tomographic image T2 on the display 51.

As a result, in the main display region 61A and the sub-display region 61B, the visibility and the usability for the user are improved as compared to a case in which the first tomographic image T1 and the second tomographic image T2 are displayed side by side in the body axis direction in a mixed manner. The reason is as follows. Since the second tomographic image T2 is an image in which a part of the region of the first tomographic image T1 is displayed, a position in the body axis direction is the same position as the corresponding first tomographic image T1. Therefore, in a case in which the first tomographic image T1 and the second tomographic image T2 are arranged in order along the body axis direction, the first tomographic image T1 and the second tomographic image T2 are mixed together. In this case, the images with different imaging ranges are mixed, so that the visibility and the usability for the user are reduced. As in the present example, the visibility and the usability for the user are improved by displaying only the first tomographic image T1 as the image displayed in the main display region 61A and the sub-display region 61B and displaying the second tomographic image T2 as necessary.

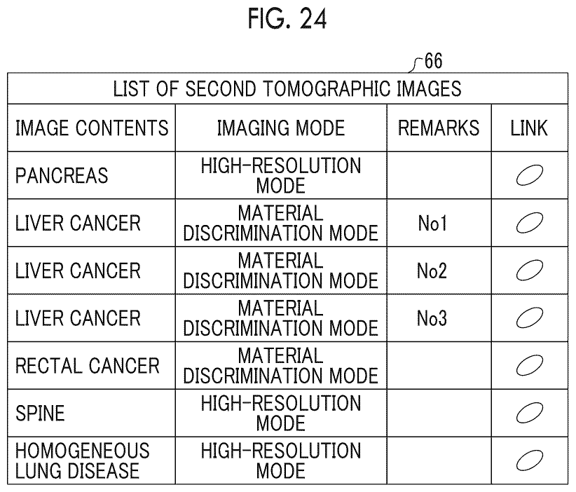

As shown in FIG. 24 as an example, a list 66 of a plurality of acquired second tomographic images T2 may be displayed. In the list 66, display fields of the image content, the imaging mode, the remarks, and the link are provided for each second tomographic image T2. In the display field of the image content, a name of the specific organ (pancreas or the like) or the abnormal part (liver cancer or the like) that is the subject of the second tomographic image T2 is displayed. The high-resolution mode or the material discrimination mode is displayed in the display field of the imaging mode. In the display field of the remarks, for example, in a case in which there is a relationship between the plurality of second tomographic images T2, such as a relationship in which one first tomographic image T1 corresponds to the plurality of specific regions AS, information (No1, No2, No3, and the like) indicating the relationship is displayed. Further, an icon of the link is displayed in the display field of the link. The second tomographic image T2 can be displayed by clicking the icon of the link.

In addition, as described above, the photon-counting detector 22 can acquire the projection data PD by distinguishing the energy bands of radiation. Therefore, with the CT apparatus 11, a virtual monochromatic X-ray image generated by dual-energy CT (DECT) technology can be obtained. The DECT technology is a technology of generating the virtual monochromatic X-ray image as captured at any single energy by combining the projection data PD captured at two tube voltages with various weightings. The virtual monochromatic X-ray image is also called a virtual monochromatic keV image.

In each of the above-described embodiments, the X-rays have been described as an example of the radiation, but γ-rays may be used as the radiation.

In addition, in the above-described embodiments, as the hardware structure of the processor 36 of the console 17 or the processor 56 of the image display apparatus 13, various processors shown later can be used. The various processors include, in addition to a CPU that is a general-purpose processor that executes software (program) to function as various processing units, a programmable logic device (PLD) of which a circuit configuration can be changed after manufacturing, such as a field-programmable gate array (FPGA), and a dedicated electric circuit that is a processor having a circuit configuration dedicatedly designed for executing specific processing, such as an application specific integrated circuit (ASIC).

Various types of processing described above may be executed by one of the various processors or may be executed by a combination of two or more processors (for example, a combination of a plurality of FPGAs or a CPU and an FPGA) of the same type or different types. Further, a plurality of processing units may be configured with one processor. As an example in which the plurality of processing units are configured with one processor, there is a form in which a processor that achieves all functions of a system including the plurality of processing units by using one integrated circuit (IC) chip is used, such as a system on a chip (SOC).

In this way, as the hardware structure, the various processing units are configured with one or more of the various processors.

Further, the hardware structure of these various processors is, more specifically, an electric circuit (circuitry) in which circuit elements such as semiconductor elements are combined.

The disclosed technology extends not only to an operation program of the CT apparatus 11 or the image display apparatus 13, but also to a non-transitory computer-readable storage medium (such as a USB memory or a digital versatile disc-read only memory (DVD-ROM)) that stores the operation program.

The above-described contents and the above-shown contents are detailed descriptions of portions related to the disclosed technology and are merely examples of the disclosed technology. For example, the description of the configuration, the function, the operation, and the effect above are the description of examples of the configuration, the function, the operation, and the effect of the parts according to the disclosed technology. As a result, it goes without saying that unnecessary parts may be deleted, new elements may be added, or replacements may be made with respect to the above-described contents and the above-shown contents within a range that does not deviate from the gist of the disclosed technology. Further, the description of, for example, common technical knowledge that does not need to be particularly described to enable the implementation of the disclosed technology is omitted in the above-described contents and the above-shown contents in order to avoid the confusion and to facilitate the understanding of the portions relating to the disclosed technology.

The technology described in the following supplementary notes can be ascertained based on the above description.

### Supplementary Note 1

A CT apparatus comprising: a photon-counting radiation detector; and a processor, in which the CT apparatus performs imaging in which projection data of radiation transmitted through a subject at a plurality of circumferential positions around the subject is detected by the radiation detector, and generates a tomographic image of the subject by reconstructing the projection data, and the processor detects a specific region that meets a predetermined condition by performing image analysis on a first tomographic image obtained by reconstructing the projection data, and generates a second tomographic image of a second imaging range that is narrower than a first imaging range of the first tomographic image and that includes the specific region.

### Supplementary Note 2

The CT apparatus according to supplementary note 1, in which the processor generates the first tomographic image by reconstructing first projection data obtained by performing the imaging in a first imaging mode as the projection data, detects the specific region by performing the image analysis on the first tomographic image, and generates the second tomographic image by reconstructing second projection data obtained by performing the imaging in a second imaging mode different from the first imaging mode.

### Supplementary Note 3

The CT apparatus according to supplementary note 2, in which in a case in which the second tomographic image is a high-resolution image having higher resolution than the first tomographic image, the second projection data is projection data for high resolution having higher resolution than the first projection data, and in a case in which the second tomographic image is a material discrimination image capable of discriminating and visualizing a plurality of materials having different attenuation coefficients of the radiation, the second projection data is projection data for material discrimination capable of being reconstructed into the material discrimination image.

### Supplementary Note 4

The CT apparatus according to supplementary note 2 or 3, in which the processor determines imaging conditions for the second imaging mode based on the specific region detected from the first tomographic image.

### Supplementary Note 5

The CT apparatus according to supplementary note 1, in which the processor reconstructs the second tomographic image using a part of the projection data used for reconstructing the first tomographic image.

### Supplementary Note 6

The CT apparatus according to supplementary note 5, in which in a case in which the second tomographic image is a high-resolution image having higher resolution than the first tomographic image, the projection data is projection data for high resolution capable of being reconstructed into the second tomographic image, and in a case in which the second tomographic image is a material discrimination image capable of discriminating and visualizing a plurality of materials having different attenuation coefficients of the radiation, the projection data is projection data for material discrimination capable of being reconstructed into the material discrimination image.

### Supplementary Note 7

The CT apparatus according to supplementary note 6, in which in a case in which the projection data is the projection data for high resolution or the projection data for material discrimination, the processor performs, in a case of generating the first tomographic image, data reduction processing on the projection data and then reconstructs the first tomographic image based on the projection data on which the data reduction processing has been performed.

### Supplementary Note 8

The CT apparatus according to any one of supplementary notes 1 to 7, in which the predetermined condition is that the specific region includes a specific organ or an abnormal part.

### Supplementary Note 9

The CT apparatus according to any one of supplementary notes 1 to 8, in which the processor performs the image analysis using a machine learning model.

### Supplementary Note 10

The CT apparatus according to any one of supplementary notes 1 to 9, in which the processor generates, in a case in which a plurality of the specific regions are detected, the second tomographic image for each specific region.

### Supplementary Note 11

The CT apparatus according to any one of supplementary notes 1 to 10, in which the processor generates a third tomographic image of a third imaging range that is narrower than the second imaging range of the second tomographic image based on a result of the image analysis of the second tomographic image.

### Supplementary Note 12

An operation method of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data, the operation method comprising: via the processor, detecting a specific region that meets a predetermined condition by performing image analysis on a first tomographic image obtained by reconstructing the projection data; and generating a second tomographic image of a second imaging range that is narrower than a first imaging range of the first tomographic image and that includes the specific region.

### Supplementary Note 13

An operation program of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data, the operation program causing the processor to execute: a step of detecting a specific region that meets a predetermined condition by performing image analysis on a first tomographic image obtained by reconstructing the projection data; and a step of generating a second tomographic image of a second imaging range that is narrower than a first imaging range of the first tomographic image and that includes the specific region.

### Supplementary Note 14

An image display apparatus that displays the first tomographic image and the second tomographic image obtained by the CT apparatus according to supplementary note 1, the image display apparatus comprising: a display control processor that controls display of the image display apparatus, in which the display control processor displays, in a case in which the first tomographic image is displayed on a display, an indicator indicating the second imaging range in the first tomographic image, and displays, in a case in which a designation operation is performed on the indicator, the second tomographic image.

In the present description, “A and/or B” is synonymous with “at least one of A or B”. That is, the expression “A and/or B” may mean only A, may mean only B, or may mean a combination of A and B. Further, in the present description, the same concept as the expression “A and/or B” is applied to a case in which the connection of three or more matters is expressed by “and/or”.

The disclosure of Japanese Patent Application No. 2023-078080, filed on May 10, 2023, is incorporated herein by reference in its entirety. In addition, all documents, patent applications, and technical standards described in the present description are incorporated herein by reference to the same extent as in a case in which the documents, patent applications, and technical standards are specifically and individually noted to be incorporated herein by reference.

What is claimed is:

1. A CT apparatus comprising:
a photon-counting radiation detector; and
a processor,
wherein the CT apparatus performs imaging in which projection data of radiation transmitted through a subject at a plurality of circumferential positions around the subject is detected by the radiation detector, and generates a tomographic image of the subject by reconstructing the projection data, and
the processor
detects a specific region that meets a predetermined condition by performing image analysis on a first tomographic image obtained by reconstructing the projection data, and
generates a second tomographic image of a second imaging range that is narrower than a first imaging range of the first tomographic image and that includes the specific region.

2. The CT apparatus according to claim 1,
wherein the processor
generates the first tomographic image by reconstructing first projection data obtained by performing the imaging in a first imaging mode as the projection data,
detects the specific region by performing the image analysis on the first tomographic image, and
generates the second tomographic image by reconstructing second projection data obtained by performing the imaging in a second imaging mode different from the first imaging mode.

3. The CT apparatus according to claim 2,
wherein in a case in which the second tomographic image is a high-resolution image having higher resolution than the first tomographic image, the second projection data is projection data for high resolution having higher resolution than the first projection data, and
in a case in which the second tomographic image is a material discrimination image capable of discriminating and visualizing a plurality of materials having different attenuation coefficients of the radiation, the second projection data is projection data for material discrimination capable of being reconstructed into the material discrimination image.

4. The CT apparatus according to claim 2,
wherein the processor determines imaging conditions for the second imaging mode based on the specific region detected from the first tomographic image.

5. The CT apparatus according to claim 1,
wherein the processor reconstructs the second tomographic image using a part of the projection data used for reconstructing the first tomographic image.

6. The CT apparatus according to claim 5,
wherein in a case in which the second tomographic image is a high-resolution image having higher resolution than the first tomographic image, the projection data is projection data for high resolution capable of being reconstructed into the second tomographic image, and
in a case in which the second tomographic image is a material discrimination image capable of discriminating and visualizing a plurality of materials having different attenuation coefficients of the radiation, the projection data is projection data for material discrimination capable of being reconstructed into the material discrimination image.

7. The CT apparatus according to claim 6,
wherein in a case in which the projection data is the projection data for high resolution or the projection data for material discrimination,
the processor performs, in a case of generating the first tomographic image, data reduction processing on the projection data and then reconstructs the first tomographic image based on the projection data on which the data reduction processing has been performed.

8. The CT apparatus according to claim 1,
wherein the predetermined condition is that the specific region includes a specific organ or an abnormal part.

9. The CT apparatus according to claim 1,
wherein the processor performs the image analysis using a machine learning model.

10. The CT apparatus according to claim 1,
wherein the processor generates, in a case in which a plurality of the specific regions are detected, the second tomographic image for each specific region.

11. The CT apparatus according to claim 1,
wherein the processor generates a third tomographic image of a third imaging range that is narrower than the second imaging range of the second tomographic image based on a result of the image analysis of the second tomographic image.

12. An operation method of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data,
the operation method comprising:
via the processor,
detecting a specific region that meets a predetermined condition by performing image analysis on a first tomographic image obtained by reconstructing the projection data; and
generating a second tomographic image of a second imaging range that is narrower than a first imaging range of the first tomographic image and that includes the specific region.

13. A non-transitory computer-readable storage medium storing an operation program of a CT apparatus including a photon-counting radiation detector, and a processor, in which the CT apparatus performs imaging in which projection data of radiation transmitted through a subject at a plurality of circumferential positions around the subject is detected by the radiation detector, and generates a tomographic image by reconstructing the projection data,
the operation program causing the processor to execute:
a step of detecting a specific region that meets a predetermined condition by performing image analysis on a first tomographic image obtained by reconstructing the projection data; and
a step of generating a second tomographic image of a second imaging range that is narrower than a first imaging range of the first tomographic image and that includes the specific region.

14. An image display apparatus that displays the first tomographic image and the second tomographic image obtained by the CT apparatus according to claim 1, the image display apparatus comprising:
a display control processor that controls display of the image display apparatus,
wherein the display control processor
displays, in a case in which the first tomographic image is displayed on a display, an indicator indicating the second imaging range in the first tomographic image, and
displays, in a case in which a designation operation is performed on the indicator, the second tomographic image.
